(19) United States
(12) Patent Application Publication
(10) Pub. No.: US 2020/0327992 A1
(43) Pub. Date: Oct. 15, 2020

(54) ARTIFICIAL INTELLIGENCE AIDED BUG BITE AND TRANSMITTED DISEASE IDENTIFICATION

(71) Applicant: Tencent America LLC, Palo Alto, CA (US)

(72) Inventors: Lianyi HAN, Palo Alto, CA (US); Nan DU, Santa Clara, CA (US); Kun WANG, San Jose, CA (US); Min TU, Cupertino, CA (US); Shangqing ZHANG, San Jose, CA (US); Hui TANG, Mountain View, CA (US); Shih-Yao LIN, Palo Alto, CA (US); Wei FAN, New York, NY (US)

(73) Assignee: Tencent America LLC, Palo Alto, CA (US)

(21) Appl. No.: 16/380,019

(22) Filed: Apr. 10, 2019

(51) Int. Cl. G16H 50/50; G06N 3/08; G16H 70/60; G16H 20/10

(52) U.S. Cl. CPC G16H 50/50; G16H 20/10; G16H 70/60; G06N 3/08

(57) ABSTRACT

A method and apparatus include receiving information associated with a bug bite. Information that identifies the bug bite is determined using a model, based on receiving the information associated with the bug bite. Information that identifies the bug bite is provided, based on determining the information that identifies the bug bite.

### BACKGROUND

Bug bites are very common and are often underestimated since insects, spiders, ticks, etc. are able to transmit myriad diseases such as chikungunya virus, yellow fever, dengue fever, Lyme disease, plague, malaria, Leishmaniasis, filariasis, etc. caused by infectious agents including viruses, bacteria, parasites, etc.

For example, Lyme disease is one of the most common vector-borne diseases in the United States, and is caused by spirochete bacteria and transmitted primarily by Ixodes scapularis (the deer tick). However, misdiagnosis is frequent and constitutes the most common cause of failure of Lyme disease treatment. In other cases, Lyme disease is simply overlooked or goes unreported. Lyme disease is a major public health issue with a significantly high price tag in terms of health care resources. Improving public awareness of bug bites that require medical attention continues to be a challenge.

### SUMMARY

According to some possible implementations, a method includes receiving, by a device, information associated with a bug bite; determining, by the device and using a model, information that identifies the bug bite, based on receiving the information associated with the bug bite; and providing, by the device, the information that identifies the bug bite, based on determining the information that identifies the bug bite.

According to some possible implementations, a device comprises at least one memory configured to store program code; and at least one processor configured to read the program code and operate as instructed by the program code, the program code including: receiving code that is configured to cause the at least one processor to receive information associated with a bug bite; determining code that is configured to cause the at least one processor to determine, using a model, information that identifies the bug bite, based on receiving the information associated with the bug bite; and providing code that is configured to cause the at least one processor to provide the information that identifies the bug bite, based on determining the information that identifies the bug bite.

According to some possible implementations, a non-transitory computer-readable medium stores instructions, the instructions comprising: one or more instructions that, when executed by one or more processors of a device, cause the one or more processors to: receive information associated with a bug bite; determine, using a model, information that identifies the bug bite, based on receiving the information associated with the bug bite; and provide the information that identifies the bug bite, based on determining the information that identifies the bug bite.

### BRIEF DESCRIPTION OF THE DRAWINGS

FIG. 1 is a diagram of an overview of an example implementation described herein;

FIG. 2 is a diagram of an example environment in which systems and/or methods, described herein, may be implemented;

FIG. 3 is a diagram of example components of one or more devices of FIG. 2; and

FIG. 4 is a flow chart of an example process for identification of bug bites using artificial intelligence (AI) techniques.

### DETAILED DESCRIPTION

Recent advances in imaging and text analysis using deep neural network (DNN) approaches have made significant progress in demonstrating artificial intelligence (AI) performance beyond that of human capability. Additionally, the availability of computationally powerful mobile devices permits AI-based applications that can be deployed and reachable by the general public.

Some implementations herein permit mobile devices to be used as assistive medical diagnosis tools. For example, some implementations herein permit the identification of a type of bug bite and/or a disease based on an input image and symptoms, and provide further suggestions regarding medical attention, available medical facilities and clinics that are accessible nearby, and/or the like.

In this way, some implementations herein permit, among other things, the education regarding and prevention of bug transmitted diseases, identification of skin lesions and symptoms caused by bug bites, reduction in the misdiagnosis of bug bites, the improvement in response time, and/or the like.

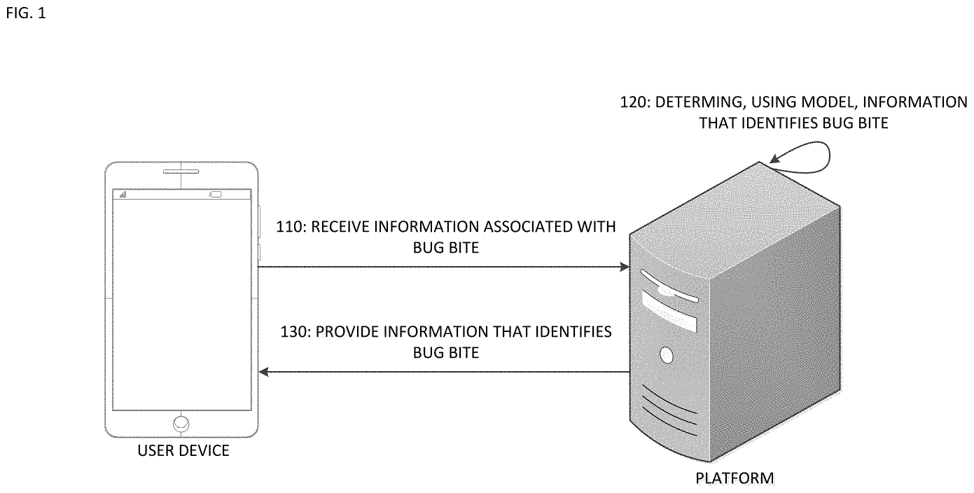

FIG. 1 is a diagram of an overview of an embodiment described herein. As shown in FIG. 1, and by reference number 110, a platform (e.g., a server) may receive, from a user device (e.g., a smart phone), information associated with a bug bite.

The user device may execute an application that permits a bug bite to be identified. In this case, assume that a user of the user device sustains a bug bite, and wishes to identify the type of bug bite, symptoms of the bug bite, a severity of the bug bite, treatment options of the bug bite, and/or the like. In this case, the user may use the application to identify the foregoing information.

For example, the user may, using the user device, capture an image of the bug bite, and input the image to the application that is being executed by the user device. The user may also enter other information such as symptoms of the bug bite, personal information, medical information, geographical information, temporal information, and/or the like. After the information is input, the user device may provide, to the platform, the information associated with the bug bite.

As further shown in FIG. 1, and by reference number 120, the platform may determine, using a model, information that identifies the bug bite. For example, the platform may input the information associated with the bug bite into the model, and determine the information that identifies the bug bite based on an output of the model. In this case, the information that identifies the bug bite may identify the type of bug bite, identify the severity of the bug bite, identify a treatment option for the bug bite, identify a nearby medical facility, and/or the like.

As further shown in FIG. 1, and by reference number 130, the platform may provide, to the user device, the information that identifies the bug bite. In this case, the user device may receive the information that identifies the bug bite, and provide, for display via a user interface (UI), the information that identifies the bug bite. In this way, the user may view the information and identify the type of the bug bite, a treatment option, etc.

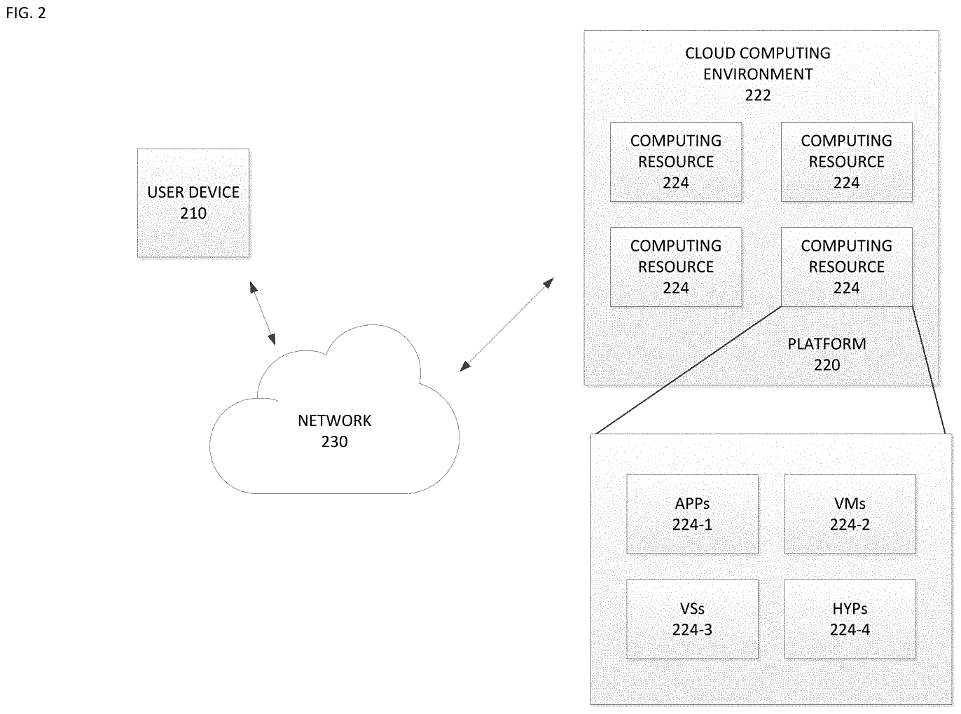

FIG. 2 is a diagram of an example environment 200 in which systems and/or methods, described herein, may be implemented. As shown in FIG. 2, environment 200 may include a user device 210, a platform 220, and a network 230. Devices of environment 200 may interconnect via wired connections, wireless connections, or a combination of wired and wireless connections.

User device 210 includes one or more devices capable of receiving, generating, storing, processing, and/or providing information associated with platform 220. For example, user device 210 may include a computing device (e.g., a desktop computer, a laptop computer, a tablet computer, a handheld computer, a smart speaker, a server, etc.), a mobile phone (e.g., a smart phone, a radiotelephone, etc.), a wearable device (e.g., a pair of smart glasses or a smart watch), or a similar device. In some implementations, user device 210 may receive information from and/or transmit information to platform 220.

Platform 220 includes one or more devices capable of identification of bug bites using artificial intelligence (AI) techniques, as described elsewhere herein. In some implementations, platform 220 may include a cloud server or a group of cloud servers. In some implementations, platform 220 may be designed to be modular such that certain software components may be swapped in or out depending on a particular need. As such, platform 220 may be easily and/or quickly reconfigured for different uses.

In some implementations, as shown, platform 220 may be hosted in cloud computing environment 222. Notably, while implementations described herein describe platform 220 as being hosted in cloud computing environment 222, in some implementations, platform 220 is not be cloud-based (i.e., may be implemented outside of a cloud computing environment) or may be partially cloud-based.

Cloud computing environment 222 includes an environment that hosts platform 220. Cloud computing environment 222 may provide computation, software, data access, storage, etc. services that do not require end-user (e.g., user device 210) knowledge of a physical location and configuration of system(s) and/or device(s) that hosts platform 220. As shown, cloud computing environment 222 may include a group of computing resources 224 (referred to collectively as “computing resources 224” and individually as “computing resource 224”).

Computing resource 224 includes one or more personal computers, workstation computers, server devices, or other types of computation and/or communication devices. In some implementations, computing resource 224 may host platform 220. The cloud resources may include compute instances executing in computing resource 224, storage devices provided in computing resource 224, data transfer devices provided by computing resource 224, etc. In some implementations, computing resource 224 may communicate with other computing resources 224 via wired connections, wireless connections, or a combination of wired and wireless connections.

As further shown in FIG. 2, computing resource 224 includes a group of cloud resources, such as one or more applications (“APPs”) 224-1, one or more virtual machines (“VMs”) 224-2, virtualized storage (“VSs”) 224-3, one or more hypervisors (“HYPs”) 224-4, or the like.

Application 224-1 includes one or more software applications that may be provided to or accessed by user device 210 and/or sensor device 220. Application 224-1 may eliminate a need to install and execute the software applications on user device 210. For example, application 224-1 may include software associated with platform 220 and/or any other software capable of being provided via cloud computing environment 222. In some implementations, one application 224-1 may send/receive information to/from one or more other applications 224-1, via virtual machine 224-2.

Virtual machine 224-2 includes a software implementation of a machine (e.g., a computer) that executes programs like a physical machine. Virtual machine 224-2 may be either a system virtual machine or a process virtual machine, depending upon use and degree of correspondence to any real machine by virtual machine 224-2. A system virtual machine may provide a complete system platform that supports execution of a complete operating system (“OS”). A process virtual machine may execute a single program, and may support a single process. In some implementations, virtual machine 224-2 may execute on behalf of a user (e.g., user device 210), and may manage infrastructure of cloud computing environment 222, such as data management, synchronization, or long-duration data transfers.

Virtualized storage 224-3 includes one or more storage systems and/or one or more devices that use virtualization techniques within the storage systems or devices of computing resource 224. In some implementations, within the context of a storage system, types of virtualizations may include block virtualization and file virtualization. Block virtualization may refer to abstraction (or separation) of logical storage from physical storage so that the storage system may be accessed without regard to physical storage or heterogeneous structure. The separation may permit administrators of the storage system flexibility in how the administrators manage storage for end users. File virtualization may eliminate dependencies between data accessed at a file level and a location where files are physically stored. This may enable optimization of storage use, server consolidation, and/or performance of non-disruptive file migrations.

Hypervisor 224-4 may provide hardware virtualization techniques that allow multiple operating systems (e.g., “guest operating systems”) to execute concurrently on a host computer, such as computing resource 224. Hypervisor 224-4 may present a virtual operating platform to the guest operating systems, and may manage the execution of the guest operating systems. Multiple instances of a variety of operating systems may share virtualized hardware resources.

Network 230 includes one or more wired and/or wireless networks. For example, network 230 may include a cellular network (e.g., a fifth generation (5G) network, a long-term evolution (LTE) network, a third generation (3G) network, a code division multiple access (CDMA) network, etc.), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the Internet, a fiber optic-based network, or the like, and/or a combination of these or other types of networks.

The number and arrangement of devices and networks shown in FIG. 2 are provided as an example. In practice, there may be additional devices and/or networks, fewer devices and/or networks, different devices and/or networks, or differently arranged devices and/or networks than those shown in FIG. 2. Furthermore, two or more devices shown in FIG. 2 may be implemented within a single device, or a single device shown in FIG. 2 may be implemented as multiple, distributed devices. Additionally, or alternatively, a set of devices (e.g., one or more devices) of environment 200 may perform one or more functions described as being performed by another set of devices of environment 200.

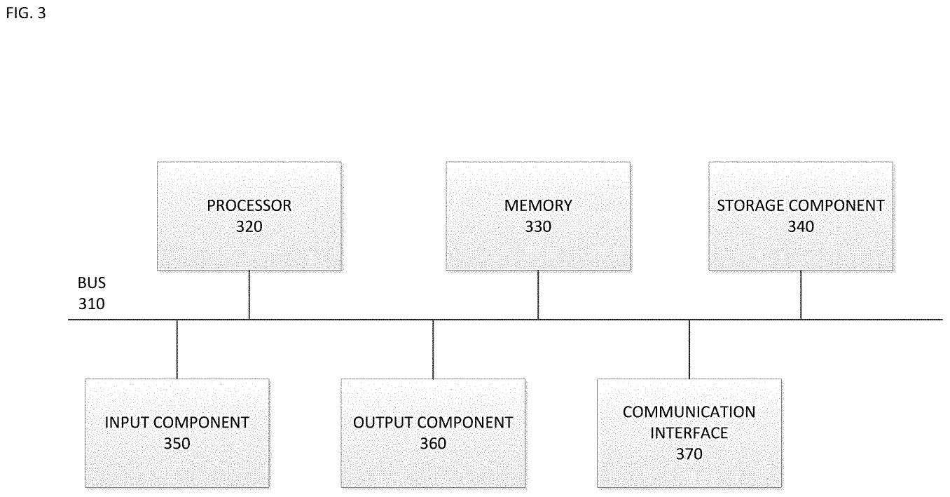

FIG. 3 is a diagram of example components of a device 300. Device 300 may correspond to user device 210 and/or platform 220. As shown in FIG. 3, device 300 may include a bus 310, a processor 320, a memory 330, a storage component 340, an input component 350, an output component 360, and a communication interface 370.

Bus 310 includes a component that permits communication among the components of device 300. Processor 320 is implemented in hardware, firmware, or a combination of hardware and software. Processor 320 is a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. In some implementations, processor 320 includes one or more processors capable of being programmed to perform a function. Memory 330 includes a random access memory (RAM), a read only memory (ROM), and/or another type of dynamic or static storage device (e.g., a flash memory, a magnetic memory, and/or an optical memory) that stores information and/or instructions for use by processor 320.

Storage component 340 stores information and/or software related to the operation and use of device 300. For example, storage component 340 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

Input component 350 includes a component that permits device 300 to receive information, such as via user input (e.g., a touch screen display, a keyboard, a keypad, a mouse, a button, a switch, and/or a microphone). Additionally, or alternatively, input component 350 may include a sensor for sensing information (e.g., a global positioning system (GPS) component, an accelerometer, a gyroscope, and/or an actuator). Output component 360 includes a component that provides output information from device 300 (e.g., a display, a speaker, and/or one or more light-emitting diodes (LEDs)).

Communication interface 370 includes a transceiver-like component (e.g., a transceiver and/or a separate receiver and transmitter) that enables device 300 to communicate with other devices, such as via a wired connection, a wireless connection, or a combination of wired and wireless connections. Communication interface 370 may permit device 300 to receive information from another device and/or provide information to another device. For example, communication interface 370 may include an Ethernet interface, an optical interface, a coaxial interface, an infrared interface, a radio frequency (RF) interface, a universal serial bus (USB) interface, a Wi-Fi interface, a cellular network interface, or the like.

Device 300 may perform one or more processes described herein. Device 300 may perform these processes in response to processor 320 executing software instructions stored by a non-transitory computer-readable medium, such as memory 330 and/or storage component 340. A computer-readable medium is defined herein as a non-transitory memory device. A memory device includes memory space within a single physical storage device or memory space spread across multiple physical storage devices.

Software instructions may be read into memory 330 and/or storage component 340 from another computer-readable medium or from another device via communication interface 370. When executed, software instructions stored in memory 330 and/or storage component 340 may cause processor 320 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of components shown in FIG. 3 are provided as an example. In practice, device 300 may include additional components, fewer components, different components, or differently arranged components than those shown in FIG. 3. Additionally, or alternatively, a set of components (e.g., one or more components) of device 300 may perform one or more functions described as being performed by another set of components of device 300.

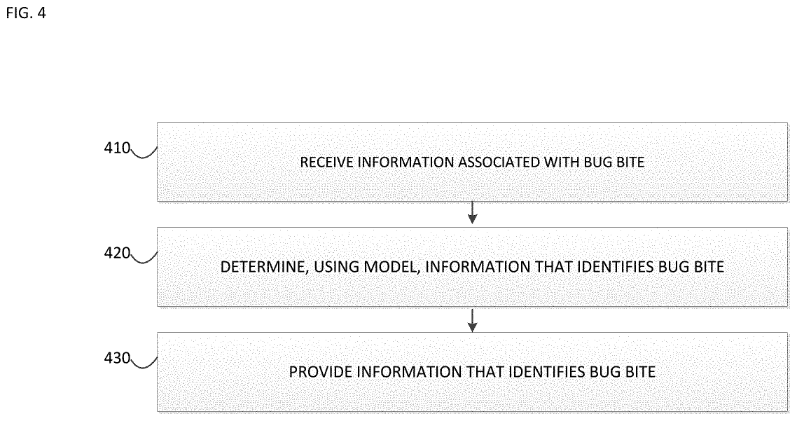

FIG. 4 is a flow chart of an example process 400 for identification of bug bites using artificial intelligence (AI) techniques. In some implementations, one or more process blocks of FIG. 4 may be performed by platform 220. In some implementations, one or more process blocks of FIG. 4 may be performed by another device or a group of devices separate from or including platform 220, such as user device 210.

As shown in FIG. 4, process 400 may include receiving information associated with a bug bite (block 410). The information associated with the bug bite may include information that permits an identification of the bug bite using an AI technique.

The information associated with the bug bite may include information about the bug bite. For example, the information associated with the bug bite may include an image of the bug bite. Additionally, or alternatively, the information associated with the bug bite may include text information associated with the bug bite. For example, the text information may include a description of a sensory reaction to the bug bite (e.g., “itchy,” “sore,” “red,” “burning sensation,” etc.), a severity of pain associated with the bug bite (e.g., “low,” “medium,” “high,” “1,” 10,” etc.), a visual description of the bug bite (e.g., “small,” “round,” “bumps,” etc.), and/or the like.

The information associated with the bug bite may include information about the circumstances of the bug bite. For example, the information associated with the bug bite may include information that identifies a geographical location where the bug bite was sustained (e.g., a country, a state, a city, specific coordinates, an event, etc.), a temporal identifier at which the bug bite was sustained (e.g., “March,” “nighttime,” “16:00,” “summer,” etc.), and/or the like.

The information associated with the bug bite may include information about the person, animal, etc. that sustained the bug bite. For example, the information associated with the bug bite may include an age of a person, an ethnicity, an occupation, a gender, allergy information, medical information, biometric information, and/or the like.

In some implementations, user device 210 may execute an application that permits the identification of bug bites using artificial intelligence (AI) techniques. For example, user device 210 may execute an application that permits a user to input the information associated with the bug bite, that determines the information the information that identifies the bug bite, and that provides the information that identifies the bug bite.

In this case, user device 210 may execute the application, and provide, for display, a user interface (UI) that permits a user to input the information associated with the bug bite. For example, a user may select an image of the bug bite, may input text information, may select from predefined selection items, and/or the like. It should be understood that any and all permutations of the information associated with the bug bite may be input via any number or types of interfaces. In any event, user device 210 may receive the information associated with the bug bite based on a user interaction with user device 210.

In this way, information that identifies the bug bite may be determined using a model, based on the received information associated with the bug bite, as described below.

As further shown in FIG. 4, process 400 may include determining, using a model, information that identifies the bug bite (block 420). For example, the information associated with the bug bite may be input to a model, and the information that identifies the bug bite may be determined based on an output of the model.

The information that identifies the bug bite may identify a type of bug associated with the bug bite. Additionally, or alternatively, the information that identifies the bug bite may identify a severity of the bug bite (e.g., “harmless,” “dangerous,” “severe,” etc.). Additionally, or alternatively, the information that identifies the bug bite may identify a set of symptoms associated with the bug bite. Additionally, or alternatively, the information that identifies the bug bite may identify a treatment of the bug bite (e.g., how to treat the bug bite).

The information that identifies the bug bite may identify a treatment location (e.g., a hospital, a clinic, and/or the like) and/or information associated with the treatment location (e.g., contact information, directions, etc.).

In some implementations, platform 220 may determine the information that identifies the bug bite based on a model. For example, platform 220 may use machine learning techniques to analyze data (e.g., training data, such as historical data, etc.) and create models. The machine learning techniques may include, for example, supervised and/or unsupervised techniques, such as artificial networks, Bayesian statistics, learning automata, Hidden Markov Modeling, linear classifiers, quadratic classifiers, decision trees, association rule learning, or the like.

The model may receive the information associated with the bug bite as an input, and generate the information that identifies the bug bite as an output. For example, the model may receive an image and descriptive symptoms of the bug bite, and determine a type of the bug bite based on the input information. It should be understood that any permutation of the information associated with the bug bite may be input, or omitted from being input, to the model.

The model may be updated (e.g., re-trained) based on receiving updated information. For example, the model may receive feedback information that identifies whether the bug bite was accurately identified. In this way, the training strategy may use transferred learning, data augmentation, strong regularization, etc. to avoid overfitting, to improve accuracy, and/or the like.

In some implementations, platform 220 may determine the information that identifies the bug bite using a model. For example, platform 220 may store the model that is used to determine the information that identifies the bug bite. In this case, platform 220 may receive, from user device 210, the information associated with bug bite. Further, platform 220 may input the information associated with the bug bite into a model, and determine the information that identifies the bug bite based on an output of the model.

In other implementations, user device 210 may determine the information that identifies the bug bite using a model. For example, user device 210 may store the model that is used to determine the information that identifies the bug bite. In this case, user device 210 might have previously received the model from platform 220. For example, platform 220 may train the model, and provide the trained model to user device 210.

After the information that identifies the bug bite is determined, the information may be provided as described below in association with block 430.

As further shown in FIG. 4, process 400 may include providing the information that identifies the bug bite (block 430).

In some implementations, user device 210 may provide, for display via a UI, the information that identifies the bug bite. In this way, a user may view the information and identify the bug bite, treatment instructions, a nearby facility, and/or the like.

In this way, some implementations herein permit a bug bite to be determined using AI techniques and input information such as an image of the bug bite and/or a description of the bug bite. In this way, some implementations herein permit more accurate identification of bug bites, improve treatment of bug bites, and/or the like.

Although FIG. 4 shows example blocks of process 400, in some implementations, process 400 may include additional blocks, fewer blocks, different blocks, or differently arranged blocks than those depicted in FIG. 4. Additionally, or alternatively, two or more of the blocks of process 400 may be performed in parallel.

The foregoing disclosure provides illustration and description, but is not intended to be exhaustive or to limit the implementations to the precise form disclosed. Modifications and variations are possible in light of the above disclosure or may be acquired from practice of the implementations.

As used herein, the term component is intended to be broadly construed as hardware, firmware, or a combination of hardware and software.

It will be apparent that systems and/or methods, described herein, may be implemented in different forms of hardware, firmware, or a combination of hardware and software. The actual specialized control hardware or software code used to implement these systems and/or methods is not limiting of the implementations. Thus, the operation and behavior of the systems and/or methods were described herein without reference to specific software code—it being understood that software and hardware may be designed to implement the systems and/or methods based on the description herein.

Even though particular combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Furthermore, as used herein, the term “set” is intended to include one or more items (e.g., related items, unrelated items, a combination of related and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

What is claimed is:

1. A method, comprising:
receiving, by a device, information associated with a bug bite;
determining, by the device and using a model, information that identifies the bug bite, based on receiving the information associated with the bug bite; and
providing, by the device, the information that identifies the bug bite, based on determining the information that identifies the bug bite.

2. The method of claim 1, wherein the information associated with the bug bite includes an image of a the bug bite.

3. The method of claim 1, wherein the information associated with the bug bite includes information that identifies a symptom.

4. The method of claim 1, wherein the information associated with the bug bite includes a location of a user associated with the bug bite.

5. The method of claim 1, wherein the information associated with the bug bite includes an age of a user associated with the bug bite.

6. The method of claim 1, wherein the model is a deep neural network (DNN) model.

7. The method of claim 1, wherein the information that identifies the bug bite includes a diagnosis of the bug bite.

8. The method of claim 1, further comprising:
identifying information that identifies a treatment for the bug bite, based on determining the information that identifies the bug bite; and
providing the information that identifies the treatment for the bug bite.

9. The method of claim 1, wherein the model includes a convolutional neural network layer.

10. The method of claim 1, further comprising:
identifying information that identifies a treatment facility, based on determining the information that identifies the bug bite; and
providing the information that identifies the treatment facility.

11. A device, comprising:
at least one memory configured to store program code;
at least one processor configured to read the program code and operate as instructed by the program code, the program code including:
receiving code that is configured to cause the at least one processor to receive information associated with a bug bite;
determining code that is configured to cause the at least one processor to determine, using a model, information that identifies the bug bite, based on receiving the information associated with the bug bite; and
providing code that is configured to cause the at least one processor to provide the information that identifies the bug bite, based on determining the information that identifies the bug bite.

12. The device of claim 11, wherein the information associated with the bug bite includes an image of a the bug bite.

13. The device of claim 11, wherein the information associated with the bug bite includes information that identifies a symptom.

14. The device of claim 11, wherein the information associated with the bug bite includes a location of a user associated with the bug bite.

15. The device of claim 11, wherein the information associated with the bug bite includes an age of a user associated with the bug bite.

16. The device of claim 11, wherein the model is a deep neural network (DNN) model.

17. The device of claim 11, wherein the information that identifies the bug bite includes a diagnosis of the bug bite.

18. The device of claim 11, further comprising:
identifying information that identifies a treatment for the bug bite, based on determining the information that identifies the bug bite; and
providing the information that identifies the treatment for the bug bite.

19. The device of claim 11, wherein the model includes a convolutional neural network layer.

20. A non-transitory computer-readable medium storing instructions, the instructions comprising: one or more instructions that, when executed by one or more processors of a device, cause the one or more processors to:
receive information associated with a bug bite;
determine, using a model, information that identifies the bug bite, based on receiving the information associated with the bug bite; and
provide the information that identifies the bug bite, based on determining the information that identifies the bug bite.
